(19) United States
(12) Patent Application Publication
(10) Pub. No.: US 2012/0016428 A1
(43) Pub. Date: Jan. 19, 2012

(54) BUNION CORRECTION METHOD AND DEVICE

(75) Inventors: Patrick Michel White, West Chester, PA (US); Thomas Wade Fallin, North Logan, UT (US)

(73) Assignee: MTP SOLUTIONS, LLC, North Logan, UT (US)

(21) Appl. No.: 13/169,861

(22) Filed: Jun. 27, 2011

Related U.S. Application Data
(60) Provisional application No. 61/359,185, filed on Jun. 28, 2010.
(60) Provisional application No. 61/365,043, filed on Jul. 16, 2010.

(51) Int. Cl. A61B 17/56

(52) U.S. Cl. 606/86 R

(57) ABSTRACT

In one aspect of the invention, a method includes locating an anchor attachment point on a first elongated bone with a first bone axis, locating a second anchor attachment point on a second elongated bone with a second bone axis, the second bone axis being misaligned relative to the first bone axis and the bones being separated by an articulating joint. The method further includes mounting a tensioning implant between the two attachment points and biasing the first and second

### FIELD OF THE INVENTION

This invention relates to a minimally invasive surgical method and device for correction of misaligned finger and toe joints.

### BACKGROUND

A bunion also referred to as Hallux Valgus is a common disease affecting the foot and is usually caused when the big toe points toward the second toe. Bunions are a progressive disorder. They begin with a leaning of the big toe, gradually changing the angle of the bones over the years and slowly producing the characteristic bump on the inside edge of the toe, which becomes increasingly prominent. Symptoms usually appear at later stages, although some people never have symptoms. Bunions occur more commonly in women and can sometimes run in families. People born with abnormal bones in their feet are more likely to form a bunion. Wearing narrow toed, high-heeled shoes may also lead to the development of a bunion. The condition may become painful as extra bone and a fluid-filled sac grow at the base of the big toe. During the early stages patients are recommended to wear slightly wider shoes and eliminate wearing high heels. In many instances however there are no simple solutions and if the bunion becomes severe, surgery may be indicated. One surgical solution is to remove a part of the metatarsal head, the part of the foot that is bulging out. This procedure is called an exostectomy or bunionectomy and is usually accompanied with soft tissue and ligament realignment around the big toe joint. Most commonly a surgeon will remove a small wedge of bone from the foot in a metatarsal or phalangeal osteotomy to achieve the realignment of the bones. Unfortunately the osteotomy is an open procedure and requires a recovery period of up to twelve weeks with the patient only partially weight bearing after several weeks since the osteotomy effectively cuts the bone in half. Due to the open nature of the surgery all the soft tissue in the joint space must heal which also slows the recovery. In another typical procedure the head of the metatarsal is resected and reshaped through a procedure called a resection arthroplasty; this is also done through an open incision. Removal of cartilage within the joint often results in a temporary fix. It is very difficult to get repeatable results while reshaping the three dimensional joint surfaces. Alternatively after the metatarsal resection there are a number of products available to replace this joint either through articulating joint replacement or spacers. While joint replacement devices increase the repeatability of the procedure, they generally do not fix the alignment and in some instances increase the probability of wear debris generation. Due to the open nature of the incision from this procedure it also requires up to a twelve week recovery time with partial weight bearing after several weeks. The last known major treatment involves fusion of the joint using pins, screws and sometimes plates to immobilize the joint. Again this is an open procedure requiring extensive healing time. Although the loss of motion in these joints can be tolerated, fusion is normally a last ditch procedure since it is currently irreversible.

Recently, a technique has been developed which involves drilling through the first and second metatarsals and anchoring them together with a suture. This technique draws the two bones together forcing the first metatarsal back into alignment.

### SUMMARY

The present invention provides a method and device for correcting misaligned finger and toe joints

In one aspect of the invention, a method includes locating an anchor attachment point on a first elongated bone with a first bone axis, locating a second anchor attachment point on a second elongated bone with a second bone axis, the second bone axis being misaligned relative to the first bone axis and the bones being separated by an articulating joint. The method further includes mounting a tensioning implant between the two attachment points and biasing the first and second axes into alignment.

In another aspect of the invention, the first bone is a metatarsal of the foot and the second bone is a proximal phalanx. The method further includes positioning an anchor at the first and second attachment points. The method further includes mounting a suture between the two anchors and tensioning the suture to draw the two bones into alignment. The suture is then fixed to maintain the alignment while allowing the joint to move through a full range of motion.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a top, or dorsal, view of a pair of feet a person with a normal left foot and a right foot with a bunion foot with a bunion;

FIG. 2 is a top cutaway view of the pair of feet of FIG. 1 revealing the bones of the forefoot;

FIG. 3 is a top cutaway view of the right foot of FIG. 1 detailing the affected joint;

FIG. 4 is a top cutaway view of the right foot of FIG. 1 showing the metatarsal after resection;

FIG. 5 is a top cutaway view of the right foot of FIG. 1 showing the bones after surgically re-alignment;

FIG. 6 is a top cutaway view of the right foot of FIG. 1 showing an operative step in correcting the alignment;

FIG. 7 is a top cutaway view of the right foot of FIG. 1 showing an operative step in correcting the alignment;

FIG. 8 is a top cutaway view of the right foot of FIG. 1 showing an operative step in correcting the alignment;

FIG. 9 is a top cutaway view of the right foot of FIG. 1 showing an alternative operative step in correcting the alignment; and

FIG. 10 is a top cutaway view of the right foot of FIG. 1 showing an alternative operative step in correcting the alignment.

FIG. 11 is a top cutaway view of the right foot of FIG. 1 showing an alternative operative step.

### DESCRIPTION OF THE ILLUSTRATIVE EXAMPLES

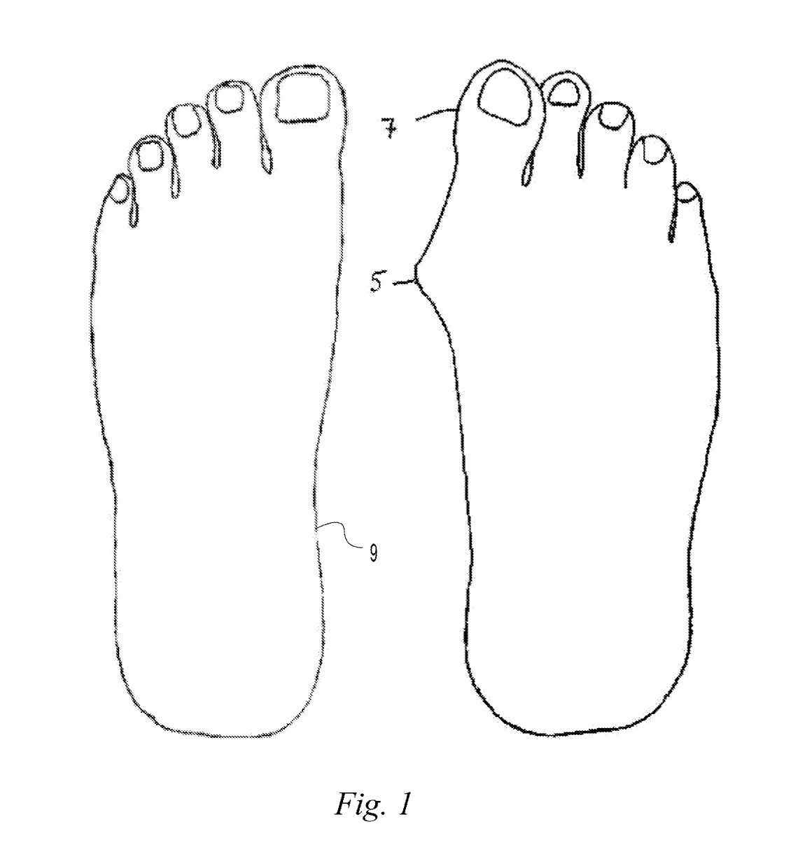

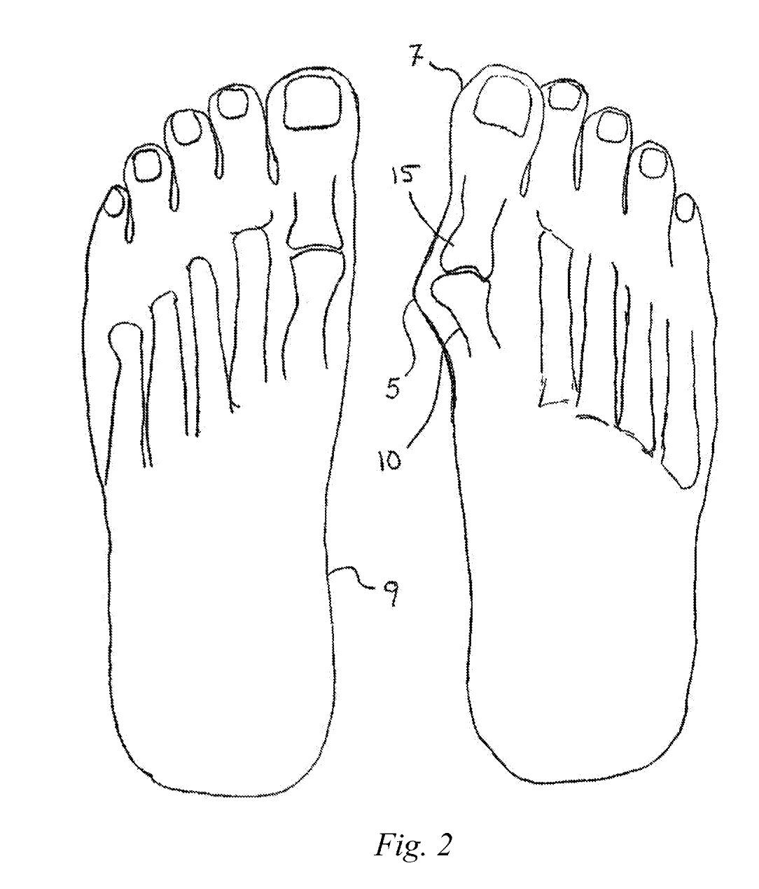

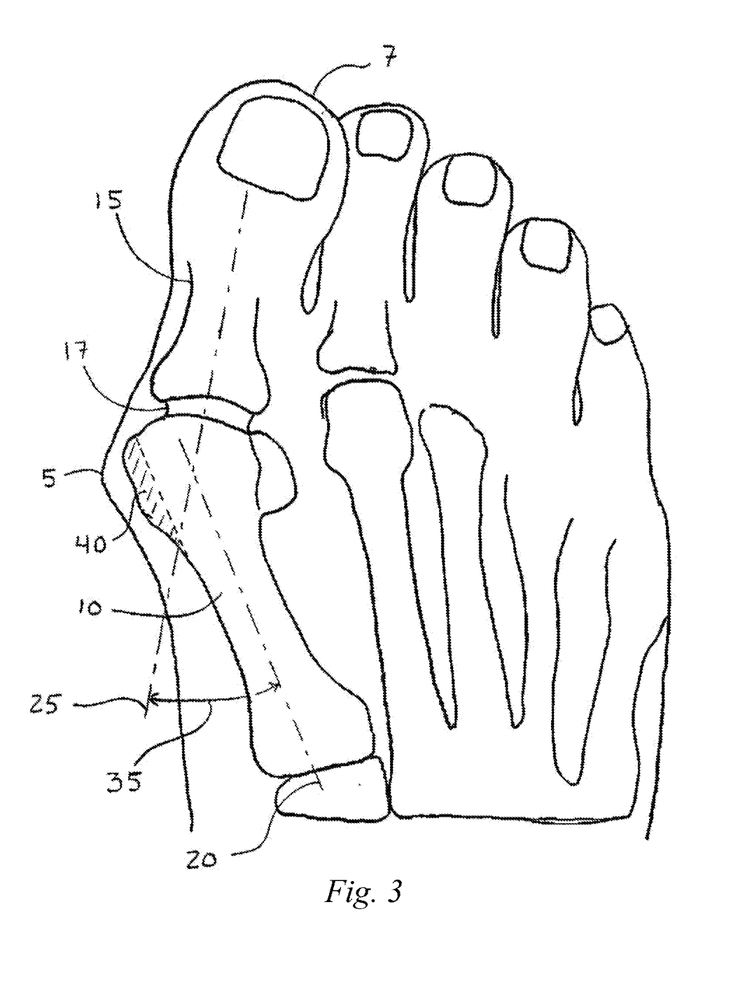

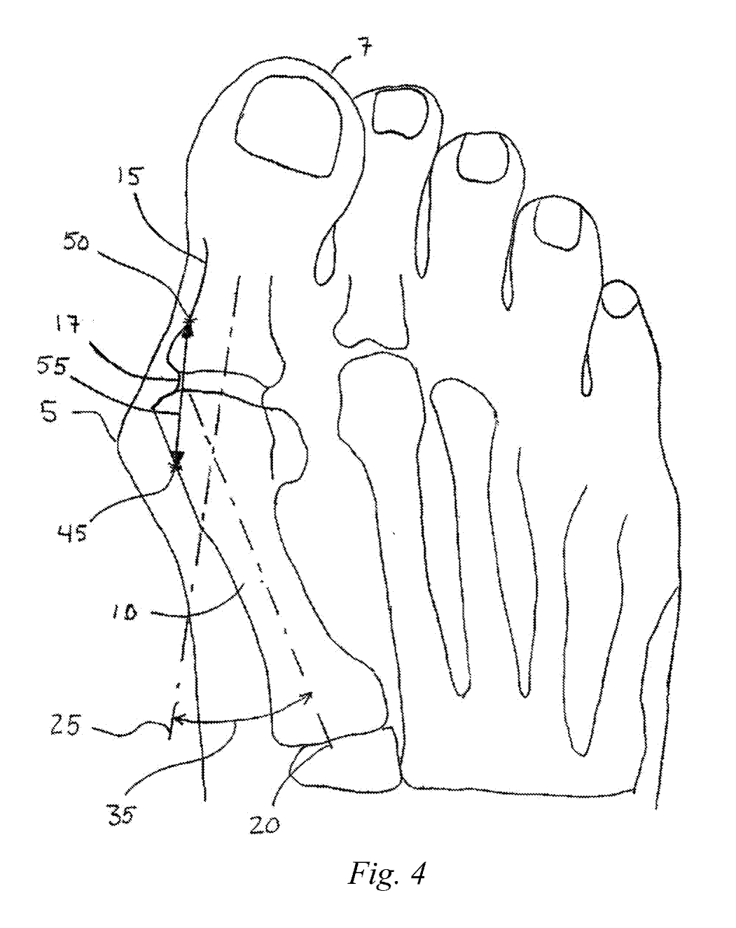

FIGS. 1-4 depict the feet of a person suffering from Hallux Valgus. The prominent bunion 5 is visible on the affected foot 7 in comparison with a normal foot 9. The bunion is formed by the misaligned joint 17 between the metatarsal bone 10 and phalangeal bone 15. The metatarsal bone 10 has an axis 20 and the phalangeal bone 15 has an axis 25 that are angled 35 in relationship to one another. Often calcifications 40 occur on the side of the metatarsal bone 10. These calcifications 40 should be removed during the procedure as depicted in FIG. 4, e.g., by sawing or chiseling the calcification off of the bone. A phalangeal attachment point 50 is located on the phalangeal bone 15 and a metatarsal attachment point 45 is located on the metatarsal bone 10. The attachment points 45, 50 are separated by an initial separation distance 55.

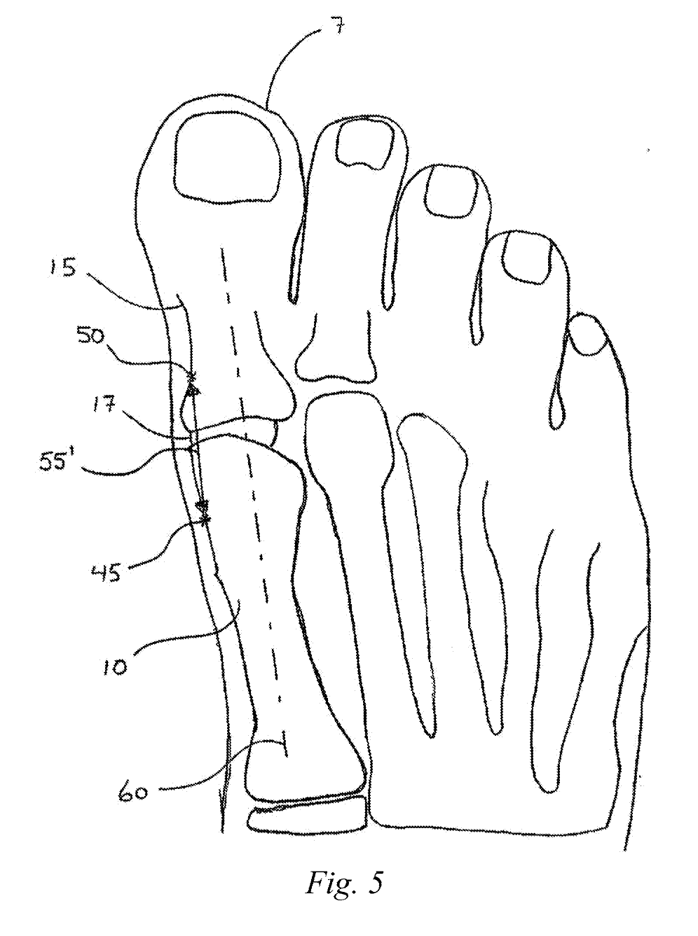

FIG. 5 illustrates that tensioning the attachment points 45, 50 toward one another shortens the initial separation distance 55 to a final separation distance 55′. The joint 17 acts as a pivot point biasing the metatarsal axis 20 and the phalangeal axis 25 towards a common axis 60, realigning the joint.

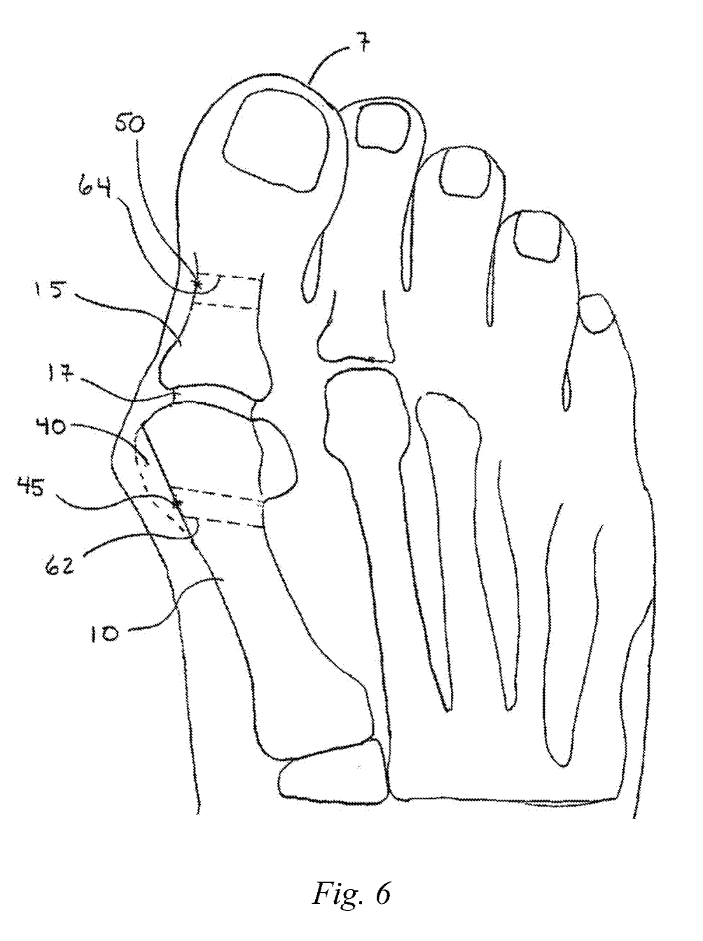

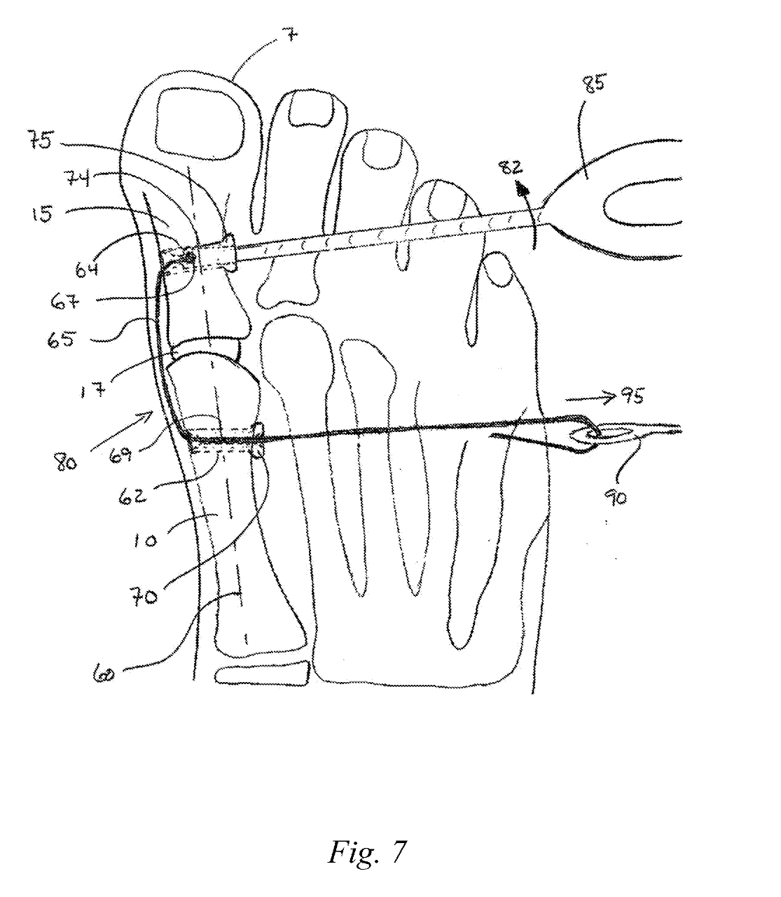

FIGS. 6-8 illustrate some of the surgical steps required to implement the implantable device 80 of the present invention. FIG. 6 shows a first hole 64 drilled through the phalangeal bone 15 at the phalangeal attachment point 50. A second hole 62 is drilled through the metatarsal bone 10 at the metatarsal attachment point 45. In addition the calcification 40 on the medial side of the metatarsal bone 10 is removed as part of the correction. FIG. 7 shows an intermediate operative step with a first anchor 74 with a flange 75 inserted into the phalangeal bone 15 through the first hole 64. A second anchor 69 with a flange 70 is inserted into the metatarsal bone 10 through the second hole 62. These holes 62, 64 are preferably drilled through the bone allowing the anchors 69, 74 to be passed across the bone, however the most important point is to create an attachment location for the tensioning cord 65 and any suitable through or blind anchors may be used. Such anchors could also be in the form of a screw, a rivet, a pin, a staple and/or a soft tissue anchor such as is used in knee or shoulder surgery.

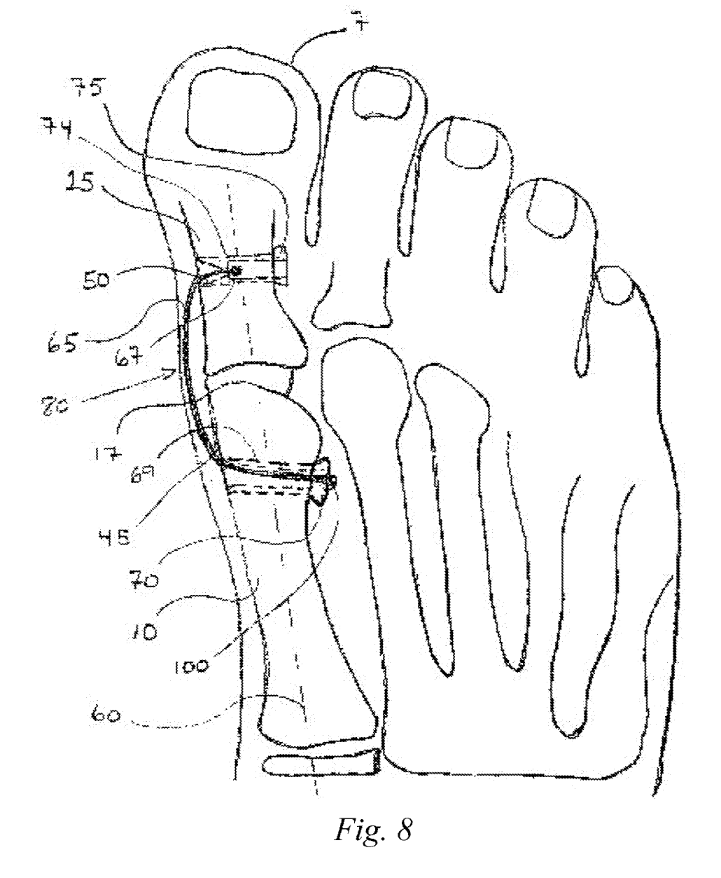

These anchors can be coated with in-growth materials to promote bonding with the one. Some typical materials might include porous coating, plasma spray coating, hydroxyapatite, or calcium phosphates. Further the anchor may be made from a porous metal or plastic foam or any surface that aids in promoting stability for the tensioning cord 65. The cord 65 may be in the form of a wire, a cable, a polymer suture, a strand, an artificial ligament, an allograft ligament, or an autograft ligament. The most important aspect of the cord 65 is its ability to tension the joint 17 while still allowing natural motion in the metatarsal phalangeal joint. In the preferred embodiment the cord 65 of the implantable device 80 is fixedly attached to the anchor 74 either through a knot 67 or crimp or any other method of securing it to the anchor. Anchor 74 may be mounted to an insertion and leverage tool 85 and urged in a direction 82 in order to assist with the pre-alignment of the phalangeal bone 15 with the metatarsal bone 10 along the common axis 60. The cord 65 may be threaded through the second anchor 69 with a needle 90 and tensioned in another direction 95. By pre-aligning with the leverage tool 85 the proper alignment can be maintained while securing the opposite end of the cord 65 to the second anchor 69. FIG. 8 shows the cord 65 of the implantable device 80 after it has been fixedly attached to the second anchor 69.

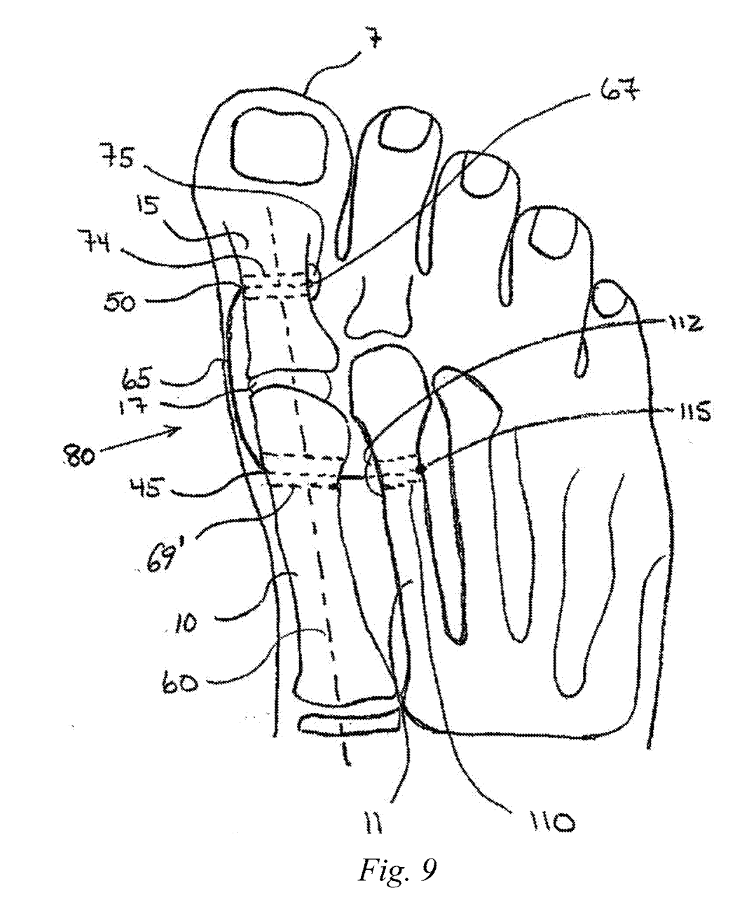

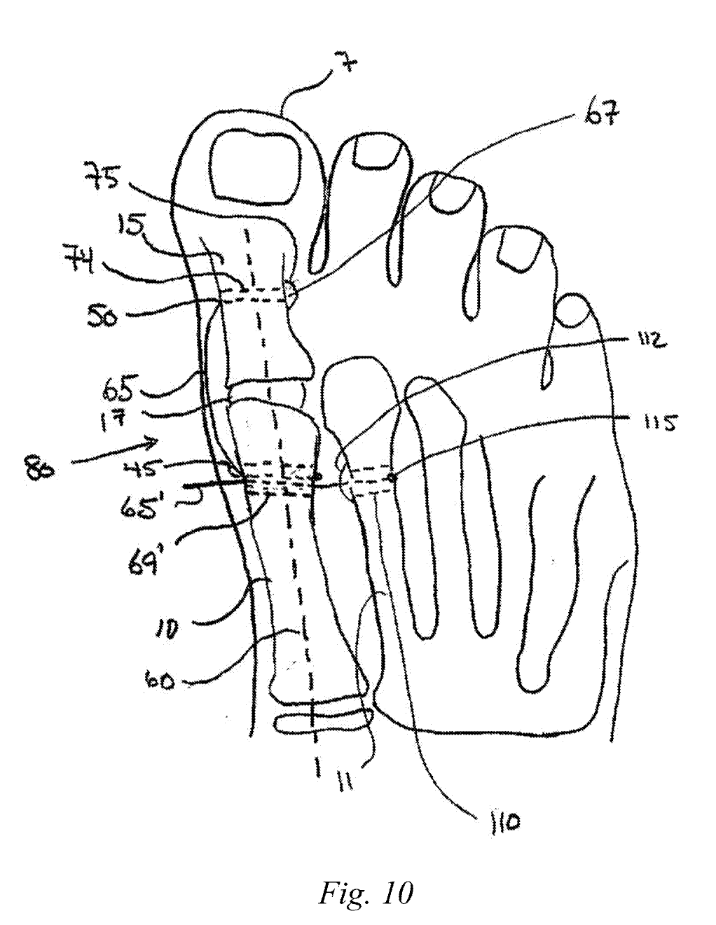

Alternate embodiments are show in FIGS. 9 and 10. Often when the hallux valgus angle requires extreme correction, the first metatarsal bone 10 must be drawn toward the second metatarsal bone 11. In order to accomplish this task the surgeon will release the sesamoid ligament and the cord 65 can either be extended between the first toe metatarsal 10 and second toe metatarsal 11 as shown in FIG. 9 or a separate cord 65′ can be extended across the toes between anchor 69′ and anchor 110, as shown in FIG. 10. In either case the goal is to hold the intermetatarsal angle between the metatarsals in an anatomic range. FIG. 10 shows the end of the cord 65′ extending from the foot. This is an optional feature where the surgeon can make small angular corrections each day by adjustably tensioning the cord 65′ over time allowing the soft tissues to adjust to the narrowed angulations. This is similar to an Ilizarov technique used to adjust bone spacing through external fixation over a period of time.

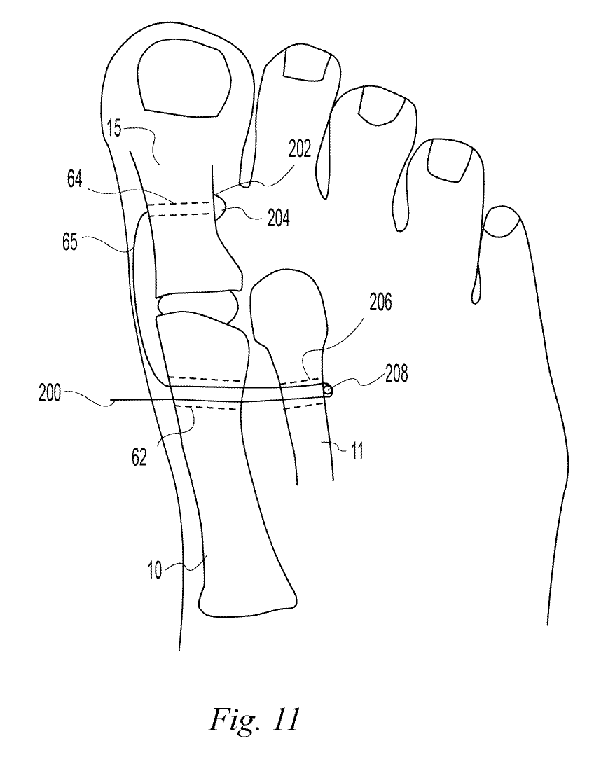

FIG. 11 illustrates another way to route the cord 65. The cord 65 has a first end 200 and a second end 202 with a preformed stopper 204, e.g. a solid mushroom shaped cap. The first end 200 is pulled through the phalangeal hole 64 from lateral to medial then through the first metatarsal hole 62 from medial to lateral. The cord is pulled through a hole 206 in the second metatarsal 11 and around a trunnion 208 (e.g. a pin, button, eyelet, pulley, or the like), back through the hole 206 in the second metatarsal 11, and back through the hole 62 in the first metatarsal 10 to extend on the medial side of the first metatarsal 10. Alternatively, the trunnion may be located on the medial side of the second metatarsal in which case not tunnel through the second metatarsal is necessary. Applying tension to the first end 200 pulls the stopper 204 into abutment with the lateral side of the proximal phalanx 15, tensions the medial side of the metatarsophalangeal joint to align the axes of the first metatarsal 10 and first proximal phalanx 15, and draws the first and second metatarsals 10, 11 together to decrease the angle between them. The cord routing of FIG. 11 allows the surgical procedure to be performed from the medial side of the foot with only small puncture wounds being required to thread the cord 65 and place the trunnion 208.

By way of example, the foregoing has described a correction for hallux valgus. However, the invention is not limited specifically to hallux valgus. Angular deformities between other small bones of the extremities may be treated with the devices and methods described herein. Variations and alternative embodiments are to be considered within the scope and spirit of the subject invention, which is to be limited only by the claims which follow and their equivalents.

What is claimed is:

1. A method of correcting hallux valgus misalignment of a first toe metatarsophalangeal joint, the joint comprising a metatarsal, a proximal phalanx, and a joint space between them, the bones and joint having a medial side and a lateral side, the method comprising:
locating a first attachment point on the medial side of the first metatarsal;
locating a second attachment point on the medial side of the first proximal phalanx;
mounting a cord between the first and second attachment points on the medial side of the metatarsophalangeal joint;
aligning the first metatarsal and first proximal phalanx in a desired alignment that reduces the valgus misalignment; and
fixing the cord to maintain the desired alignment.

2. The method of claim 1 wherein the step of locating a first attachment point includes forming a transverse hole through the first metatarsal and the step of locating a second attachment point includes forming a transverse hole through the first proximal phalanx.

3. The method of claim 2 wherein the step of aligning the first metatarsal and first proximal phalanx in a desired alignment that reduces the valgus misalignment includes tensioning the cord to draw the bones into alignment.

4. The method of claim 2 wherein the step of aligning the first metatarsal and first proximal phalanx in a desired alignment that reduces the valgus misalignment includes:
manual manipulation of the bones into a desired alignment; and
tensioning the cord to maintain the manual alignment.

5. The method of claim 4 wherein the step of aligning the first metatarsal and first proximal phalanx in a desired alignment that reduces the valgus misalignment includes engaging one of the bones with a lever in torque transmitting relationship and moving the lever to align the bones.

6. The method of claim 5 wherein engaging one of the bones with a lever includes inserting a lever into the bone hole formed in the first proximal phalanx.

7. The method of claim 6 wherein the hole through the proximal phalanx has a medial entrance and a lateral entrance, the method further comprising placing an anchor sleeve in the lateral entrance and engaging the anchor sleeve with the lever.

8. The method of claim 2 wherein the bone transverse holes each have a medial entrance and a lateral entrance, the method further comprising:
placing an anchor sleeve in the lateral entrance of each hole; and
engaging the cord with the anchoring sleeve to anchor the cord to the bone.

9. The method of claim 2 further comprising:
mounting a cord between the first and second metatarsals;
aligning the first and second metatarsals; and
fixing the cord to maintain the desired alignment.

10. The method of claim 9 wherein the cord between the first and second metatarsals is the same cord that connects the first and second attachment points, the method further comprising:
locating a third attachment point on the medial side of the second metatarsal and extending the cord between the second and third attachment points.

11. The method of claim 10 further comprising:
extending the cord back to the first metatarsal and out the medial side of the first metatarsal; and
pulling on the cord on the medial side of the first metatarsal to tension the cord and align the first and second metatarsals and the first metatarsal and first proximal phalanx.

12. The method of claim 11 wherein the cord has a preformed stopper at one end, the method further comprising:
pulling the cord through the hole in the first proximal phalanx from lateral to medial so that the stopper abuts the lateral hole entrance.

13. The method of claim 9 wherein the cord between the cord between the first and second metatarsals is a different cord than the one that connects the first and second attachment points.

14. The method of claim 1 wherein the tensioning cord is selected from the list consisting of wires, cables, polymer sutures, strands, artificial ligaments, allograft ligaments, and autograft ligaments.

15. The method of claim 1 wherein the tensioning cord is connected to the selected attachment points through an anchor.

16. The method of claim 3 wherein the anchors are selected from the list consisting of screws, rivets, pins, staples, and soft tissue anchor.

17. The method of claim 4 wherein the anchors have a bone growth interface selected from the list consisting of porous coatings, plasma spray metal coatings, hydroxyapatite, calcium phosphate, metal foam, and plastic foam.
